(19) United States
(12) Patent Application Publication
(10) Pub. No.: US 2021/0287057 A1
(43) Pub. Date: Sep. 16, 2021

(54) METHOD OF PROCESSING DATA TO IMPROVE MOTION RECOGNITION, ASSOCIATED SENSOR AND SYSTEM

(71) Applicant: OCTONION SA, Lausanne (CH)

(72) Inventors: Fernando RAMAO, Paris (FR); Andrei SHELEH, Minsk (BY); Ivan SELIVANAU, Brest (BY)

(21) Appl. No.: 16/319,627

(22) PCT Filed: Jul. 17, 2017
(86) PCT No.: PCT/EP2017/068060

§ 371 Date: Jan. 22, 2019

(30) Foreign Application Priority Data
Jul. 22, 2016 (FR) 1657076

(51) Int. Cl. G06K 9/66; G06K 9/00; G06F 16/2458; H04L 29/08

(52) U.S. Cl. CPC G06K 9/66; H04L 67/12; G06F 16/2462; G06K 9/00342

(57) ABSTRACT

Disclosed is a system and a method for processing data so as to improve motion recognition implemented by an application associated with at least one data sensor. Raw data are collected by the at least one sensor and stored in a remote database. An analysis, preferably, statistical of all or part of the raw data stored in the database is performed by a server linked to the database. The application is parametrized and/or updated as a function of the results of the analysis. The system includes one or more data sensors, the server and the remote database and a mobile terminal.

### FIELD OF THE INVENTION

The present invention concerns the field of movement recognition implemented by data sensors.

The invention is particularly applicable in the field of sport to recognize the movements made by a sportsperson in different disciplines, such as golf, tennis or skiing.

In this field of application, there are data sensors qualified as “multisport” that are intended for the evaluation of the performance of a sportsperson during his or her practice of sport, it being possible to configure these sensors according to the sport that is practiced.

For example, in a tennis training session, a data sensor fastened to the wrist of a sportsperson must enable identification of the number of forehand and backhand strokes made by that player. For this, reliable recognition of the movements made by the player is necessary.

In view of the multiplicity of sports practiced throughout the world, there is a need to efficiently configure or adapt the movement recognition algorithms implemented on each sensor, to improve the reliability of recognition and thereby increase the reliability of the analysis of sports performances resulting therefrom.

According to the current state of the art, it is known to perform local exploitation of raw data, such as inertial quantities measured by accelerometers and/or gyrometers built into multisport data sensors. These data are directly processed by the data sensors by means of a dedicated program employing movement recognition algorithms, these algorithms being provided to recognize the nature or type of the movements executed during practice of a sport.

The dedicated program analyzes the raw data supplied by the sensors and produces what are referred to as reduced data which contain the recognized movement and possibly associated measurements. For example, the reduced data may contain the information that the recognized movement is a forehand stroke in tennis associated with the speed of the racket at the moment of the strike. However, it may happen that the recognition of the movements by the algorithms implemented by the dedicated program is not optimum. Thus may arise from the physical or practical specificities of the user for example.

### Subject of the Invention

The present invention is directed to mitigating the aforementioned drawbacks, by providing a technical solution making it possible to improve the movement recognition implemented by data sensors, for a wide variety of sports practices.

These objectives are attained by a method of data processing configured to improve the movement recognition implemented by a program associated with at least one data sensor, said method comprising the following steps:
collecting raw data by said at least one sensor;
storing the raw data in a remote database;
performing analysis, in particular statistical, of all or part of the raw data stored in the remote database; and
parameterizing and/or updating said program according to the analysis results obtained further to the analyzing step.

The analysis, in particular statistical, of the raw data from a plurality of sensors used by different users is particularly advantageous for improving the performance in recognizing movements by a generic algorithm of the program, in particular when such an algorithm is implemented by a high number of sensors. The recourse to statistical analysis methods is all the more advantageous when the number of sensors enabling the remote database to be supplied is high.

For a given sensor, the analysis, in particular statistical, of raw data from that same sensor is also advantageous for improving the performance of a movement recognition algorithm of the program specific to that sensor, it being possible for the algorithm to be customized according to the raw data supplied by that sensor.

In all cases, it is on the basis of the raw data obtained from one or more data sensors that the recognition of movements of a sportsperson may be improved. The result of the analysis is used to parameterize or update the movement recognition algorithm or algorithms in centralized manner. This is all the more advantageous when the number of sensors employed is high. Thus, the programs for movement recognition implemented on the data sensors can be updated or configured in centralized manner, on the basis of the raw data obtained from a set of sensors.

According to a specific embodiment of the invention, the method further comprises a step of prior testing, wherein a movement recognition reliability criterion is evaluated, if a movement is recognized by the program, such that the other steps of the method are initiated according to the value of the reliability criterion relative to a predetermined threshold.

Thus, the steps of the method according to the invention are executed solely if the recognition of movements made by the data sensor has an insufficient degree of reliability. The conditional execution of the steps of the method according to the invention is particularly advantageous for avoiding any unnecessary processing and for saving the electrical resources of the data sensor of which the autonomy must be optimized.

Given that the raw data are generally voluminous, the fact of sending them on particular occasions is especially advantageous for limiting the amount of information to have to pass over radio-mobile communications networks, given the limited transmission capacities of the radio technologies currently implemented and of the massive implementation of connected data sensors.

According to a specific embodiment of the invention, an identifier qualifying the type of movement made by a user of said at least one sensor is associated with said raw data at the collecting step. The identifier is input, manually by the user or automatically, based on a predefined sequence of movements to be carried out, the identifier and the data being transmitted to said database.

The identifier advantageously makes it possible to classify and group together the raw data of the same nature (for example corresponding to the practice of the same sport in similar conditions) in the remote database.

According to a particular embodiment of the invention, the collected raw data are sent by said at least one data sensor to a mobile terminal, that terminal being adapted to transmit said data to the remote database.

The use of a mobile terminal, such a mobile telephone, to transfer raw data from the data sensor to the remote database is particularly well-suited to the case in which the sensors do not have means for communicating with the radio-mobile networks.

Thus, the collected raw data are sent to the remote database by the mobile telephone using the subscription linked to that telephone. In this case, it is possible to use sensors with a simplified architecture, of which the energy consumption is reduced.

In an example embodiment, the raw data comprise velocity and/or acceleration inertial data.

The inertial data are not only used by the movement recognition algorithm but are also exploited in the analysis step to adapt the program of the data sensor, for the purpose of improving the recognition capabilities.

Optimally, the raw data furthermore comprise at least one of the following parameters: orientation of the data sensor, ambient pressure, ambient temperature.

These additional parameters are indicators of physical conditions in which the data sensor has been used at the time of collecting the inertial data. It will be possible to take such indicators into consideration in the analysis of the collected raw data.

According to a specific embodiment, the method further comprises a merging step, wherein the collected raw data are merged, so as to provide a structure of data that are calibrated, filtered and more accurate than if they were considered in isolation

By way of a first example, the gyrometer biases (i.e. low-frequency component) are eliminated by the computation of a moving average in the static phases of the data sensor or are eliminated by more powerful algorithms such as those described by Kalman-Bucy and known by the name “Kalman filters”.

By way of a second example, the data from the gyrometer and from the accelerometer are combined, that is to say merged to attain higher accuracy in the computation of attitude (orientation in space) of the data sensor. This merging is carried out using Kalman filtering or by use of a complementary filter. These techniques are known to the person skilled in the art and therefore are not described in detail here.

Thus, the merging of the collected raw data is directed to calibrating the quantities measured when collecting data, for example, relative to a common reference value and/or when converting these data so as to express them according to a conventional system of units that can be directly interpreted in the analysis step. The calibration in particular makes it possible to compensate for static and dynamic defects by the combination of complementary data to improve the overall accuracy.

According to a specific embodiment, the parameterizing step includes the provision to said at least one data sensor of at least one configuration parameter that is determined according to the results of the analysis.

The configuration parameter or parameters provided to the data sensor or sensors are used to customize the recognition algorithm implemented by the program of that data sensor or those data sensors. The centralized provision of these configuration parameters is particularly advantageous for simultaneously customizing a plurality of implemented sensors, without having to take action individually on each of the sensors.

According to a specific embodiment, the updating step includes the provision to said at least one sensor of a program that is updated according to the results of the analysis.

Thus, centralized updating of the sensor programs may be carried out efficiently. This is particularly advantageous for generalizing a recognition algorithm (i.e. for improving a generic algorithm) on the basis of raw data, that are preferably merged, obtained from a high number of data sensors.

The invention is also directed to a method of data processing configured for improving the movement recognition implemented by a program associated with at least one data sensor. Said method comprises the following steps implemented by said at least one data sensor:
collecting raw data;
sending the collected raw data to a remote database; and
receiving configuration parameters and/or an update of said program, said parameters and/or said update being determined according to the results of processing of an analysis, preferably statistical, of all or part of the raw data stored in the remote database.

The invention is also directed to a method of data processing configured to improve the movement recognition implemented by a program associated with at least one data sensor, said method comprising the following steps implemented by a data server:
receiving raw data collected by said at least one sensor;
storing said raw data in a remote database;
performing analysis, preferably statistical, of all or part of the data contained in said database; and
parameterizing and/or updating said program according to the results of the analysis step.

The invention is also directed to a system for data processing configured for improving the movement recognition implemented by a program associated with at least one data sensor, said system comprising:
means for collecting raw data by said at least one sensor;
means for storing the raw data in a remote database;
means for analysis, preferably statistical, of all or part of the raw data stored in the database; and
means for parameterizing and/or updating said program according to the analysis parameters obtained by the analysis means.

According to a specific embodiment of the invention, the system further comprises a mobile terminal adapted to send, to the remote database, raw data received from said at least one sensor.

The invention is also directed to a data sensor for improving the movement recognition, said sensor comprising:
means for collecting raw data;
means for sending said data to a remote database;
means for receiving at least one configuration parameter and/or an update of said program, said at least one parameter and/or said update being determined according to the results an analysis, preferably statistical, of all or part of the raw data stored in the remote database.

The invention is also directed to a data server configured for improving the movement recognition implemented by a program associated with at least one data sensor, said server comprising:
means for receiving raw data obtained by measuring means of at least one data sensor;
means for storing said raw data in a database;
means for analysis, preferably statistical, of all or part of the data contained in said database; and
means for parameterizing and/or updating said program according to the results obtained by the analysis means.

According to a specific embodiment, the server is configured to implement at least one movement recognition algorithm in accordance with an algorithm implemented on said at least one data sensor configured to be parameterized or updated. Thus, the algorithm is executed by the server using all or part of the raw data stored in the database.

The invention also concerns a computer program comprising instructions adapted to the updating of any one of the steps of the methods according to the invention as described above, when said program is executed on a computer.

This program may use any programming language, and be in any form of source code, object code, or code intermediate between source code and object code, such as in a partially compiled form, or in any other desirable form.

The invention is also is directed to an information storage means, which may or may not be removable, and which is partially or totally readable by a computer or a microprocessor comprising code instructions of a computer program for executing any one of the steps of the methods according to the invention as described above.

The data carrier may be any entity or device capable of storing the program. For example, the carrier may comprise a storage means, such as a ROM (Read Only Memory), for example a microcircuit ROM, a magnetic recording means, for example a hard disk, or a flash memory.

Moreover, the data carrier can be a transmissible medium, such as an electrical or optical signal, which can be conveyed via an electrical or optical cable, or else by radio or by other means. A program according to the invention may in particular be downloaded onto a storage platform of a network of Internet type.

Alternatively, the information carrier may be an integrated circuit, in which the program is incorporated, the circuit being configured to execute or be used in the execution of the method in question.

The aforementioned data carrier and computer program have features and advantages that are similar to the methods they implement.

### BRIEF DESCRIPTION OF DRAWINGS

Still other particularities and advantages of the invention will appear in the following description, in relation with the accompanying drawings which are given by way of non-limiting examples, and in which:

FIG. 1 illustrates the architecture of a system according to a first embodiment of the invention;

FIG. 2 illustrates the architecture of a system according to a second embodiment of the invention;

FIG. 3 illustrates an example embodiment of a system according to the first embodiment of the invention comprising a plurality of sensors;

FIG. 4 illustrates the steps of the method according to a specific embodiment of the invention;

FIG. 5a illustrates an example of representation of two scatter plots respectively corresponding to two gestures, illustrating the Hamming distance; and

FIG. 5b illustrates an example of partitioning to enable movement recognition.

One of the ideas underlying the invention is to parameterize and/or update the program of one or more data sensors to improve the reliability of movement recognition, according to the results of an analysis, preferably statistical, of the raw data stored in a remote database, these data being collected from at least one data sensor.

The system according to the invention comprises at least one data sensor used by one or more users, and a server linked to the remote database. The server is configured to analyze the raw data contained in the database. The collected data may be sent to the server via a mobile terminal connected to the data sensor. In an alternative embodiment, the data sensor is adapted to send the collected data directly to the data server.

The raw data stored in the database are associated with an identifier making it possible to qualify the type of movement made on collecting corresponding raw data. Each set of raw data collected in association with the predefined movement identifier constitutes a full data block, it being possible for the set of data blocks stored in the database to be exploited by analysis methods which are preferably statistical.

The results of the analysis of the raw data are used to determine configuration parameters destined to improve the configuration of the movement recognition algorithm implemented by the program of the data sensor. Thus, the invention advantageously enables a reconfiguration of the program of each data sensor, by taking into account all or part of the raw data stored in the remote database.

The results of the analysis of the raw data may also be used to produce an update of the program of the data sensor, on which is implemented the movement recognition algorithm. Thus, the invention enables the updating of the program of one or more data sensors, by taking into account all or part of the raw data stored in the remote database obtained from the sensors.

In all cases, the configuration and/or the updating of the program of one or more data sensors are carried out in centralized manner, by means of the server, taking into account all or part of the raw data obtained from a sensor or a plurality of sensors.

Typically, the invention enables a generic movement recognition algorithm to be updated or configured, it being possible for this algorithm to be common to a plurality of sensors regularly supplying the remote database.

The purpose of the algorithms implemented on the set of sensors is not only to recognize a given movement and categorize it (for example tennis forehand stroke) in order for create statistics communicated to the user, but also to measure with the highest possible accuracy a characteristic parameter which will also be communicated to the user, such as the velocity of a movement or the height of a jump.

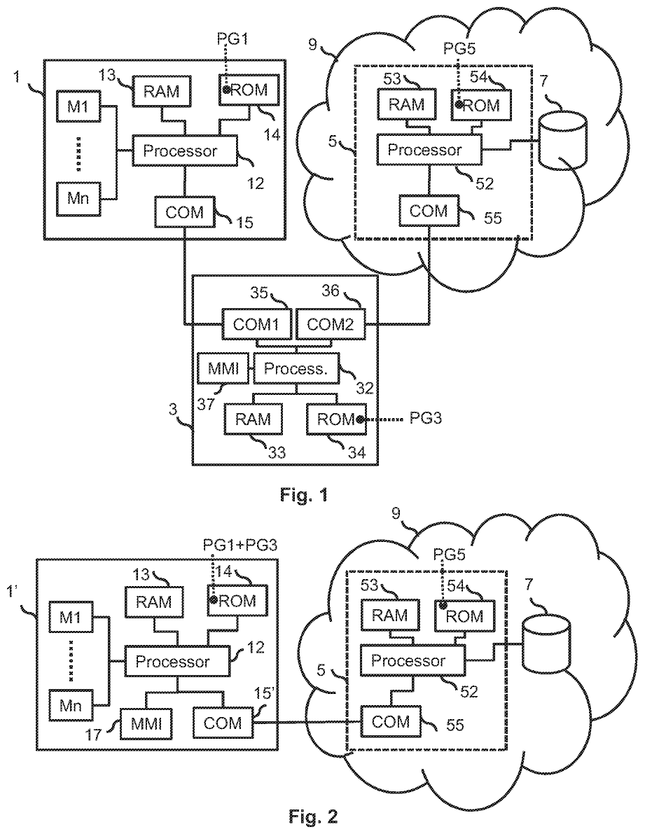

FIG. 1 diagrammatically illustrates the architecture of a system according to a first embodiment of the invention.

The system comprises:
a multisport data sensor 1,
a mobile terminal 3 constituted, for example, by a mobile telephone,
a server 5 is a remote database 7 in a telecommunications network 9.

In the present example, the telecommunications network 9 comprises the Internet network in which is included the server 5 and the remote database 7. The mobile terminal 3 is provided with 3G or 4G type radio-mobile communication means configured to access the Internet network according to IP communication protocols.

The data sensor 1 comprises measuring means M₁, . . . , Mₙ for measuring inertial quantities, such as the velocity and/or the acceleration, these measuring means being integrated into the data sensor 1.

In a particular embodiment of the invention, the data sensor 1 comprises three measuring means as follows.
first measuring means M₁ configured to measure an instantaneous angular velocity, this velocity being represented by a vector ω having three rotational velocity components ωₓ, ω_{y}, ω_{z} around each of the axes of said first measuring means M₁ constituted by a gyrometer built into said data sensor;
second measuring means M₂ configured to measure a local linear acceleration represented by a vector γ having three linear acceleration components γₓ, γ_{y}, γ_{z} along each of the axes of said second measuring means M₂, these means being constituted by a linear accelerometer built into said data sensor;
third measuring means M₃ configured to compute a Linear World Acceleration Γ based on the accelerations measured by one or more accelerometers and rotational velocities measured by one or more gyrometers, the Linear World Acceleration being computed according to algorithms known to the person skilled in the art (e.g. rotation matrices using Euler angles or quatrenions, merging of data using complementary filters, Kalman filtering), these means creating what may be named a “linear world accelerometer”.

The data sensor 1 may furthermore comprise additional measuring means, such as a pressure sensor and/or a temperature sensor.

The data sensor 1 further comprises:
a central processing unit 12 central processing unit a microprocessor, in particular of ARM type such as Cortex-M4 or a different one;
a volatile memory 13 of RAM (Random Access Memory) type and a non-volatile memory 14 of ROM (Read Only Memory) type;
a communication interface 15, for example of short-range radiofrequency type of Bluetooth® type.

All the components constituting the data sensor 1 as described above may typically be integrated onto a same single-unit printed circuit of PCB (Printed Circuit Board) type.

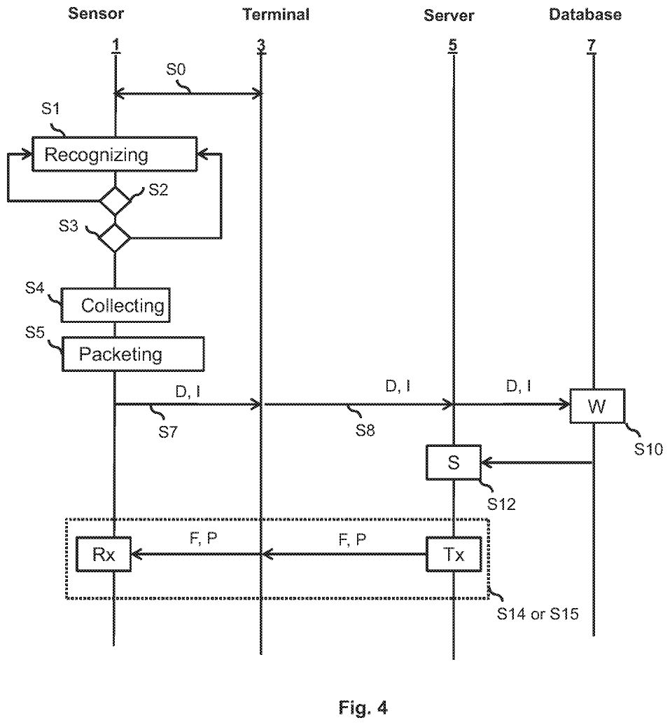

In the example described here, the non-volatile memory 14 constitutes a recording medium which stores a computer program PG1 in accordance with the invention, able to implement, when it is executed by the central processing unit 12, the steps of the method of data processing carried out by the data sensor in accordance with the invention, as illustrated in FIG. 4.

The mobile telephone 3 typically comprises:
a short-range radiofrequency communication interface 35 (COM1) of Bluetooth® type configured to communicate with the communication interface 15 (COM) of the data sensor 1;
a radio-mobile communication interface 36 (COM2) of 3G or 4G type;
a volatile memory 33 of RAM type and a non-volatile memory 34 of ROM type;
a central processing unit 32, such as a micro-processor;
a man-machine interfere 37 (MMI) comprising a display device.

In the example described here, the non-volatile memory 34 (ROM) constitutes a recording medium which stores a computer program PG3 in accordance with the invention, able to implement, when it is executed by the central processing unit 32, the steps of the method of data processing carried out by the mobile terminal 3 in accordance with the invention, as illustrated in FIG. 4.

The server 5 is linked to the database 7 on the telecommunications network 9.

The server 5 typically comprises:
a communication interface 55 (COM) configured to communicate with the radio-mobile communication interface 36 (COM2) of the mobile telephone 3;
a volatile memory 53 of RAM type and a non-volatile memory 54 of ROM type;
a central processing unit 52, such as a micro-processor.

In the example described here, the non-volatile memory 54 constitutes a recording medium which stores a computer program PG5 in accordance with the invention, able to implement, when it is executed by the central processing unit 52, the steps of the method of data processing carried out by the server 5 in accordance with the invention, as illustrated in FIG. 4.

FIG. 2 diagrammatically illustrates the architecture of a system according to a second embodiment of the invention.

This second embodiment basically differs from the first embodiment described with reference to FIG. 1, in that the data sensor 1′ is configured to communicate directly with the server 5. In this case, it is not necessary to have a mobile telephone 3, which is the case for the autonomous multi-function data sensors.

In this case, the data sensor 1′ comprises, for example, a radio-mobile communication interface 15′ (COM) of 3G, 4G and/or wireless of Wi-Fi® type configured to communicate directly with the server 5.

The data sensor 1′ further comprises a man-machine interfere 17 (MMI) comprising a display device.

In order not to needlessly encumber the present description, all the other components designated by the same reference numbers as those already described with reference to FIG. 1 remain identical and are not described again.

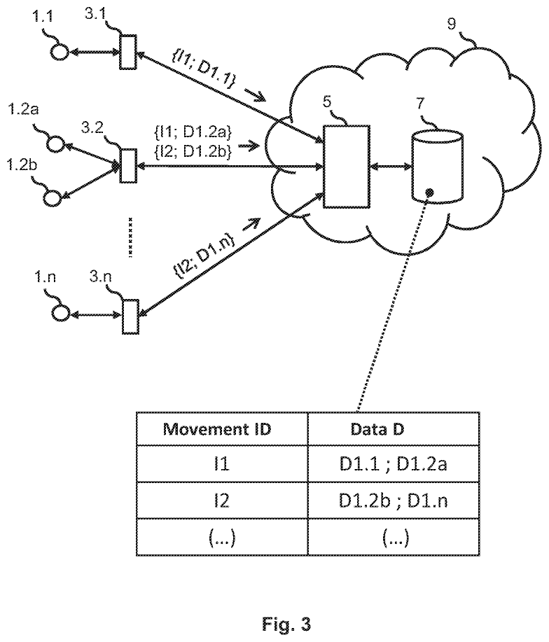

FIG. 3 illustrates an example embodiment of the system according to the first embodiment of the invention, wherein a plurality of data sensors 1.1, 1.2a, 1.2b, . . . , 1.n are configured to supply the same remote database 7 via mobile telephones 3.1, 3.2, . . . , 3.n with which they are respectively paired. It will be noted that several sensors 1.2a, 1.2b can be paired with the same mobile telephone 3.2.

FIG. 4 illustrates an example embodiment of the method according to the invention as implemented by the system illustrated in FIG. 1 according to the first embodiment.

In the interest of simplification, the method will be described with reference to a single data sensor, but will still be valid for each of the sensors of the plurality of sensors of the system illustrated in FIG. 3, in which several sensors may simultaneously or sequentially transmit raw data to the remote database 7. The server 5 and the database 7 are dimensioned appropriately according to the number of sensors and/or mobile telephones that may supply the database 7.

Initially, it is assumed that the data sensor 1 is paired with the mobile telephone 3 by means of the application PG3 executed on the telephone 3, at a prior pairing step E₀.

As soon as the data sensor 1 is paired with the telephone 3, the user can select, from the application PG3, the type of sport practiced by means of the man-machine interface 37. This information is provided to the sensor 1 which is able to configure itself according to the selected sport.

In a nominal operating mode, the data sensor 1 executes the program PG1 configured to recognize, in real time, movements made by a sportsperson in action. To that end, the processor 12 of the data sensor 1 executes a predefined movement recognition algorithm in the program PG1, at a recognition step S₁.

This recognition is carried out in real time and according to the raw data measured by the measuring means of the data sensor 1. In the present example, these raw data comprise the angular velocity values ω measured by the gyrometer M₁, the local linear acceleration values γ measured by the local linear accelerometer M₂ and the Linear World Acceleration values F supplied by the linear world accelerometer M₃.

To enable the movement recognition, the program PG1 implements a movement recognition algorithm, this algorithm being predefined on putting the sensor into operation by means of standard parameters.

However, according to a particular feature of the invention, it is possible for this algorithm to be adapted or updated according to a statistical analysis of the raw data supplied by one or more sensors as described below.

After each iteration of the recognition step S₁, the program PG1 checks the reliability of this recognition, according to at least one reliability criterion, at a first optional test step S₂. To that end, the program PG1 compares the value of said at least one recognition reliability criterion relative to a predetermined reference threshold (reliability condition).

By way of illustrative non-limiting example, the reliability criterion or indicator used is the Hamming distance. In this case, the first test step S₂ consists of computing the Hamming distance between the movement pattern recognized at a given instant and two neighboring patterns recognized previously further to the recognition step.

If the computed distance is greater than a predetermined reference threshold, the reliability condition is met and in this case, the data sensor continues the recognition S₁ in real time of the movements made by the sportsperson, in its nominal operating mode.

If the computed Hamming distance is abnormally low, that is to say if it is too often below the predetermined reference threshold, a step S₄ of collecting the raw data is initiated automatically by the data sensor 1.

For example, the step of collecting the raw data will be triggered on the condition that the Hamming distance is less than the reference threshold for more than 70% of the movements detected in a training session.

In this example, the rate of occurrence of this condition is set at 70%. It will be possible for this value to be defined based on the data collected in advance and on the analysis of the statistical distribution of the population studied (which served to build the movement recognition algorithms).

The recognition information obtained further to the recognition step S₁ is reduced data (e.g. backhand, velocity, effect) which may be transmitted to the application PG3 executed by the mobile telephone 3 to indicate to the user the movements recognized and their characteristics, by means of the man-machine interfere 37. In contrast to the raw data, the reduced data cannot be exploited in order to improve the movement recognition algorithms, since the reduced data are the result of that recognition.

Optionally, the collecting step S₄ is initiated according to the profile of the user (sportsperson) and/or his or her sporting performances, further to a second test step S₃.

For example, the collecting step S₄ may be initiated if at least any one of the following conditions is met:
the user belongs to a predetermined category of sportspersons (e.g. professional);
the Hamming distance is too often below a threshold.

Such conditions are verified by the program PG1 of the data sensor 1 at the second test step S₃, according to the user's profile information and/or performance information, it being possible for all or part of that information to be supplied by the application PG3 executed on the mobile telephone 3.

According to the type of sport selected in advance by the user, the application PG3 presents to him or her, by means of the man-machine interface 37, a sequence of predetermined movements to perform during the data collecting step S₄.

For example, in the case of tennis, the application PG3 will provide the user with instructions to perform the following sequence of movements; five backhand strokes, five forehand strokes, and three serves. Thus, the collected data may be associated with predetermined movements according to the proposed sequence.

This sequence comprises a list L of identifiers qualifying the type of movements to make. For example, this list L is pre-recorded in the program PG1 of the sensor 1. It may also be sent to the sensor by the application PG3 of the mobile telephone 3 when a sport has been selected on the application PG3. Thus, an identifier I specifying the type of movement to make is automatically associated with the collected raw data according to the movement sequence presented to the user.

According to the example illustrated in FIG. 3, it is assumed that at a given instant, a first identifier I₁ designating, for example, a tennis backhand stroke is associated with the raw data D1.1 and D1.2a obtained from the data sensors 1.1 and 1.2a further to the execution of a backhand stroke by each of the two sportspersons. These data are sent to the server 5 in association with the first identifier I₁ by the mobile telephones 3.1 and 3.2 respectively. Similarly, a second identifier I₂, designating, for example, a forehand tennis stroke is associated with the raw data D1.2b and D1.n supplied by the data sensors 1.2b and 1.n, these data being transmitted to the server 5 by the mobile telephones 3.2 et 3.n respectively;

Thus, the data D1.1 et D1.2a associated with the first identifier I₁ and the data D1.2b and D1.n associated with the second identifier I₂ are stored in the database 7.

According to a variant embodiment, the collected raw data are manually qualified by the user, for example, when the latter selects a movement in the application PG3 from a predefined list of movements before performing that movement, in a collecting step S₄. This qualification results in associating the identifier I of the movement made with the raw data collected during the performance of that movement.

In the second embodiment, in the absence of use of a mobile telephone 3, the sequence of movements is presented by means of the man-machine interfere 17 included in the data sensor 1′.

In all cases, the nature of the movement made by the sportsperson is qualified and associated with the raw data collected in performing that movement.

At the collecting step S₄, these data are recorded by the data sensor for example in the form of a local file. In the present example, the local file contains all the data measured by the gyrometer M₁, the local linear accelerometer M₂, the linear world accelerometer M₃ and possibly the pressure and temperature data obtained by pressure and temperature sensors during the collecting step S4.

The collected raw data are packeted at a packeting step S₅. The packeting of the data consists of converting the measured data into a structure of raw data calibrated relative to reference values and expressed according to units able to be interpreted for example by a human or by a statistical processing algorithm. In the present example, the packeted data constitute a raw data structure comprising:
Angular velocity data D₁ measured by the gyrometer M₁ and expressed on the basis of three orthogonal axes in a coordinate system linked to the data sensor 1;
local acceleration data D₂ measured by the local linear accelerometer M₂ and taking into account gravity and expressed in m/s² on the basis of the three axes of the accelerometer in the local coordinate system linked to the data sensor 1, and
Linear World Acceleration data D₃ measured by the linear world accelerometer M₃, not taking into account gravity and expressed in m/s² on the basis of the three axis of the accelerometer in the world coordinate system.

The packeted data may also comprise:
a special data structure qualified as “Quaternion” describing the orientation of the data sensor in space; and/or
atmospheric pressure data expressed in Pascal (Pa); and/or
atmospheric temperature data expressed in degree Celsius (° C.).

These packeted data constitute a data structure comprising inertial data and possibly raw pressure and temperature data, all these data being calibrated relative to reference values for each of the quantities concerned.

The collected and packeted raw data D are sent by the data sensor 1 to the mobile telephone 3 by means of a Bluetooth® communication, at a transmission step S₇.

For each movement made in the collecting step S₄, the packeted raw information D is sent by the communication interface 15, these data being sent in association with the identifier I enabling the nature or the type of the movement made to be identified. According to the example in FIG. 3, the packeted data D_{1.1} associated with the identifier I₁ supplied by the data sensor 1.1 are transmitted by the mobile telephone 3.1. to the data server 5.

On reception, the data D_{1.1}, D_{1.2a}, D_{1.n} are stored on the mobile telephone 3 before being transferred by it to the server 5 via the network 9, at a second transmission step S₈.

In the present example embodiment, the sending of the raw data is automatically proposed to the user on the application PG3 executed on his or her mobile telephone 3. In this case, the program PG1 executed on the data sensor 1 sends a request to the application PG3 of the mobile telephone to request the user to accept the sending of the collected raw data to the remote database 7.

According to a variant embodiment, the sending of the raw data is carried out without action by the user, for example randomly or automatically when the condition of reliability of the recognition is not met.

The random character is particularly advantageous to increase the knowledge of the set of users concerned and refine the algorithms accordingly. For example, assuming that in a particular region of the world or for a given category of persons, the movement recognition performance is less good, obtaining complementary raw data is particularly advantageous for carrying out a campaign of creation and/or modification of movement recognition algorithms. The server can contact around ten users of the category mentioned for the campaign via the application PG3 executed on their mobile telephone. In this case, a request is issued by the server to those telephones. In response to this request, the user inputs, by means of the application PG3, the identifier I making it possible to identify the nature or the type of movement made, such that the latter is transmitted in association with the raw data.

According to another variant embodiment, it is provided that the user wishing to improve the movement recognition achieved by his or her data sensor can initiate at will the steps of collecting S₄, packeting S₅ and sending S₇ of the raw data, on the basis of the application PG3 executed on his or her mobile telephone 3, without the implementation of the optional test step S₂. In this case, the user inputs the nature or the type of movement carried out, such that the latter is transmitted in association with the raw data.

In the second embodiment, in the absence of a mobile telephone, the collecting of raw data is initiated by the user by means of an interface provided on the data sensor, these data being then sent by the data sensor 1′ directly to the remote database 7. In this case, the program of the sensor includes all or part of the functionalities provided on the application PG3 of the mobile telephone described above.

In all cases, the packeted raw data are not sent continuously which enables the amount of data transmitted to be limited. This is particularly advantageous since the raw data are voluminous. For example, using an accelerometer cadenced at a sampling frequency of 500 Hz, the characterization of a movement for a duration of one second requires several kilo-bytes of data.

Sending these data at particular times enables the electricity consumption of the data sensor to be substantially reduced relative to the case in which the raw data are sent continuously. Thus, the energy autonomy of the sensor can be optimized.

Furthermore, given the proliferation of connected data sensors, it is important to take care not to needlessly congest the radio-mobile communication networks of which the transmission capacities are limited. In this connection, the sending of the raw data under conditions is advantageous.

On reception of the raw data, the server 5 stores these data in the remote database 7 at a storage step S₁₀. Given that these raw data are associated with movements that are known according to the sequence of movements presented to the user in the collecting step S₄, these constitute a set of data that can be exploited to increase the reliability of the movement recognition implemented by different data sensors.

The remote database 7 comprises an identifier of the type of movement concerned for each item of raw data coming from a sensor. For example, the database can comprise several sets of data that are characteristic of a same type of movement for a given sport. According to the example illustrated in FIG. 3, the raw data D1.2b and D1.n respectively coming from the sensors 1.2b and 1.n are stored in the database 7 in association with the same identifier I₂ designating, for example, a tennis backhand stroke.

The centralized storage in the database 7 enables all the sensors implemented to contribute to the enrichment of the content of that database at the collecting step S₄. The supply of this database in centralized manner by a high number of sensors makes it possible to constitute a set of data sufficiently complete to be statistically analyzed.

The enrichment of the content of the database makes it possible in particular to constitute an extended list of the possible ways to perform different movements.

The regular enrichment of the content of this database by raw data coming from different sensors is particularly advantageous to improve the overall reliability of the movement recognition implemented on one or more sensors.

The server is adapted to implement at least one movement recognition algorithm for each sport practiced. For this, the server is adapted to implement at least the three following modules:
an activity qualifier configured to identify the nature of a movement that is characteristic of a sports practice, by detecting the different phases of a movement based on measured raw data, a corresponding activity data stream being supplied as output from the activity qualifier;
a classifier for classifying the activity data output from the qualifier; and
an analyzer to process the classified activity data output from the classifier.

The activity qualifier supplies the classifier with an activity data stream comprising qualifier values, such as root mean square and/or average values for the acceleration and the rotational velocity along one or more axes, for example expressed in the local coordinate system of the sensor and/or in the coordinate system of the terrestrial frame of reference.

The choice of the qualifier values is made according to each sport considered, for example, further to a human analysis. The choice may be made according to tests of trial-and-error type supported by statistical correlation analyses.

For example, in the case of a movement made by a boxer, the qualifier detects, on the basis of the measured raw data, that a punch has been made by the boxer and provides the classifier with the data for acceleration along the local x-axis (component γₓ) measured by the local linear accelerometer M₂.

According to the sport considered, other examples of qualifier values can be considered, such as the amplitude of the movement and/or of the elements of projection of the movement trajectory into a reference plane.

For example, the trajectory detected by the sensor may be projected into a horizontal plane and the two dimensions (length L1 and width L2) of a rectangle in which the projected trajectory is defined will be computed. In this case, the length L1 and the width L2 of the rectangle are considered as qualifier values which proves to be very useful to establish the difference between a direct punch and a hook in boxing, for example.

Other qualifier values may also be obtained by preceding in the same way, in a plane perpendicular to the horizontal plane. In this case, the main plane of the movement is detected then the trajectory of the movement is projected onto that main plane.

According to a variant embodiment, the trajectory may be temporarily divided up into a plurality of segments, such that the qualifier values as described above are computed over each of these segments.

To ensure good recognition reliability for the movements, the classifier may be configured according to the sport practiced. This module enables pattern recognition, by the implementation of machine learning algorithms, based on qualifier values supplied by the activity qualifier. Such algorithms are, for example, based on C45 type decision trees, logistic regression or neural networks. This classification method is supervised, in the sense that the nature of the movement is known in advance for performing the classification by virtue of the activity data stream supplied by the classifier.

A linear regression algorithm may also be used to classify the different movements. Establishing classification rules may be carried out using decision tree learning methods. The decision tree so determined enables the movements to be classified, that is to say to recognize them based on a substantial database of sports gestures that have already been classified.

The result supplied by the classifier is a set of recognized movements with which are generally associated the data for acceleration and attitude of the sensor in the terrestrial frame of reference. These data are used by the analyzer implementing algorithms specific to each sport and to each movement recognized by the classifier to compute metrics that are of interest to the user, such as the speed of the racket just prior to impact with the ball in the case of tennis, the height of a jump in the case of horse-riding or skiing, or the acceleration on impact in the case of boxing. Thus at the output of the analyzer module a stream of sports movements or gestures is available, each movement being identified, dated and characterized by metrics configured for the sport practiced.

At a statistical analysis step S₁₂, the server 5 executes statistical processing of all or part of the raw data contained in the database 7 to improve the movement recognition algorithms.

To that end, the raw data are processed by the server 5 according to statistical analysis methods, so as to supply one or more data sensors with:
configuration parameters for the best-suited algorithm; or
an improved version of the algorithm.

According to the particular style of movement of a sportsperson for a given sport, the configuration of the movement recognition algorithm implemented by one or more sensors may be such that the reliability of movement classification is not optimum.

In this case, the raw data supplied by the sensor of that sportsperson and recorded in the remote database in a training session are exploited by the data server 5 to adapt the classification algorithm of the classifier and therefore the classifier implemented by the server.

The adaptation of this classifier makes it possible to improve the recognition reliability of the algorithm further to the statistical analysis of the raw data of a particular sportsperson or of a group of sportspersons, the recognition algorithm of a sensor coming from the classifier implemented by the server.

Thus, the classification algorithm may be adapted or reconfigured according to specific data of the sportsperson. This adaptation is carried out according to known statistical analysis methods to customize the recognition algorithm, for example of C4.5 type.

The statistical analysis methods advantageously enable the taking into account of a very high number of individual data which could not be processed manually by a human being. This is in particular the case for improving the recognition reliability of a generic algorithm for a sport practiced by a high number of users.

By way of illustrative example, the statistical analysis methods may exploit the criterion of the Hamming distance to reconfigure or adapt the classification algorithm of the classifier module implemented on the server. Thus, the movement recognition algorithm is re-configured or adapted, such that the Hamming distance is the greatest possible to limit the risk of confusing movements.

The adaptation of the classification algorithm of the classifier module according to the Hamming distance by statistical analysis methods constitutes a means for determining configuration parameters of the algorithm making it possible, for example, to maximize the Hamming distance for an optimum recognition of movements.

By way of example, it is assumed that in the movement recognition algorithm initially implemented on the sensors, only two Qualifiers are used and that the minimum Hamming distance found for the whole population of sportspersons is equal to 0.25 (normalized Hamming distance). If a particular sportsperson (i.e. not included in the initial population) presents a Hamming distance equal to 0, there is a case for attempting to improve the conditions for recognizing the gestures of that sportsperson.

In the context of boxing, the inventors initially found that the Hamming distance was 0.25 by considering the two following Qualifiers: “Variance of the acceleration along the local x-axis” and “Variance of the rational velocity around the local z-axis”. Next, by performing more complete tests with new types of sportspersons, this distance passed to 0 indicating confusion for the recognition of certain gestures, this being due to a more pronounced rotational movement around the local x-axis.

In this case, the correction or adaptation of the algorithm consists of replacing the previous Qualifiers with the “Variance of the acceleration in the terrestrial fixed plane x,y” and the “Variance of the rotational velocity around the terrestrial fixed z-axis”, in order to see the Hamming distance normalized for all the sportspersons (including the particular sportsperson) to a value equal to 0.3. This is typically the type of situation which may arise for a new sportsperson with a very particular style.

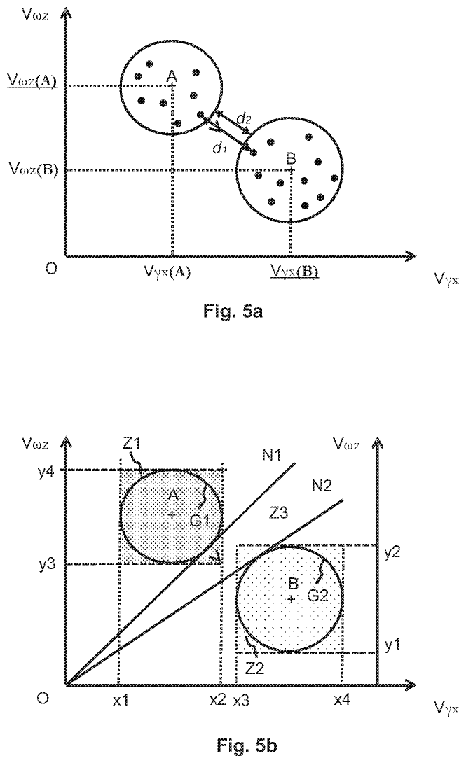

All or part of the raw data stored in the remote database may be presented in the form of scatter plots of measurement on a two-dimensional diagram, according to the example illustrated in FIG. 5a, in which the axis for the abscissae represents the variance of the linear average acceleration along the local x-axis (Vγx) and the axis for the ordinates represents the root mean square value of the rotational speed around the local z-axis (Vωz). According to this representation, a first scatter plot G1 is centered on a first reference point A of coordinates (Vγx(A), Vωz(A)) designating the center of gravity relative to a first gesture, whereas a second scatter plot G2 is centered on a second reference point B of coordinates ((Vγx(B), Vωz(B)) designating the center of gravity relative to a second gesture.

The Hamming distance found for a population of sportspersons tested is illustrated by the vector dl. In this representation, the normalized Hamming distance is used. To express the normalized distances, the values Vωz along the axis for the ordinates are divided by the value of the ordinate Vωz(A) of the first reference point A and the values Vγx along the axis for the abscissae are divided by the value of the abscissa Vγx(B) of the second reference point B. Normalization facilitates the comparison of performance of different algorithms.

Further to the analysis S12 of the collected raw data, the movement recognition algorithm used by the data sensor or sensors that supplied the raw data is adapted, so as to maximize the Hamming distance characterizing the difference in signature of each movement.

For example, attention is turned to the Hamming distance between two types of detected movement characterized by two parameters, such as the average acceleration and the root mean square of the rotational speed around an axis.

In this example, the statistical analysis method implemented by the server 7 comprises:
a partitioning step which consists of defining, in the representation of FIG. 5a, three non-contiguous zones Z1, Z2, Z3 according to the example illustrated in FIG. 5b: a first zone Z1 corresponding to a first gesture, a second zone Z2 corresponding to the second gesture, and a third intermediate zone Z3 located between the first and second scatter plots;
a test step which consists of verifying whether each of the points of measurement belongs to one of the three predefined zones.

In the present example, the partitioning is defined as follows:
the first zone Z1 is defined as the intersection of two straight lines x1, x2 parallel to the axis of the ordinates and two straight lines y3, y4 parallel to the axis of the abscissae;
the second zone Z2 is defined as the intersection of two straight lines x3, x4 parallel to the axis of the ordinates and two straight lines y1, y2 parallel to the axis of the abscissae;
the third zone Z3 is defined by the surface comprised between a first straight line N1, which is a tangent to the first scatter plot G1 and passes through the point of origin O formed by the intersection of the axis of the abscissae and the axis of the ordinates, and a second straight line N2, which is a tangent to the second scatter plot G2 and passes through the point of origin O, these two straight lines forming a minimal angle between them.

In the example described above, the configuration parameters of the movement recognition algorithm are the following:
the variance of the acceleration in a terrestrial fixed plane X, Y (Vγ_{x,y}) and the variance of the rotational speed around the z-axis (Vωz) being selected as adapted qualifiers or qualifier values,
the parameters x1, x2, y3, y4 defining the first zone Z1, the parameters x3, x4, y1, y2 defining the second zone Z2, and the two tangent straight lines N1, N2 defining the third zone Z3.

According to the positioning of the values measured by the accelerometer and the gyrometer on these zones, it will be possible to distinguish the movements between them. In the interest of simplicity, only two qualifier values were used in the above example. However, in practice, five or six qualifier values may be used to characterize each of the movements.

A user having movements of which the recognition too often leads to points that are close (i.e. small Hamming distance, for example equal to 0) will be judged less reliable than a person producing movements with a large Hamming distance. A small Hamming distance constitutes an indicator of reduced recognition reliability.

The statistical analysis of the collected raw data leads to increasing the Hamming distance, which results in making the movement recognition achieved by the movement recognition algorithm more reliable.

The statistical analysis algorithms may be supplied by more qualifier values potentially making it possible to increase the Hamming distance to further improve the recognition reliability.

According to the results of the statistical analysis carried out on the collected raw data, it is determined whether improvements may be made to the movement recognition algorithm or to the firmware of the data sensor including the recognition algorithm.

In the case of supervised learning, the adaptation of the classifier at the server may be assisted by a human being who defines in advance the types of movement considered for the statistical analysis. If the movement has been classified in advance by the user, the classifier module implemented at the server may operate autonomously and the firmware may thus be modified or generated automatically. Human intervention is provided to verify proper operation and to authorize the official distribution of the modified or generated firmware.

Below, firmware will be used to designate the program of the data sensor, this program containing one or more movement recognition algorithms, and other functions such as the management of charging of the battery, display management and management of Bluetooth management.

It is assumed that the raw data come from a same data sensor used by a same sportsperson for the movement recognition of a given sport. In this case, the statistical analysis step advantageously makes it possible to extract configuration parameters intended for configuring the program executed on the data sensor according to the specificities of the sensor and/or of the sportsperson considered.

Assuming that the raw data come from a plurality of data sensors used by different sportspersons for the movement recognition of a given sport, the statistical analysis step advantageously makes it possible to extract updated parameters that are useful for evolving the program implemented on all the sensors concerned.

At an updating step S₁₄, a new firmware version is generated which is intended for one or more sensors requiring such an update, according to the results of the statistical analysis step S₁₂, it being possible for this generation to be implemented by the server, with or without the intervention of a human being in particular for needs of control or verification.

By way of illustrative example, the value of the Hamming distance resulting from the implementation of the classification algorithm of the classifier module by the server 5 constitutes a result enabling a modification of the firmware to be decided. Thus, an update may be made according to the Hamming distance computed in the statistical analysis step relating to all or part of the data stored in the database. In this respect, the Hamming distance constitutes a parameter potentially triggering an update of the firmware.

In case of modification, the new firmware is sent by the server 5 to the mobile telephone 3 which transmits it to the data sensor or sensors 1 concerned by that update. The installation of the new firmware on the data sensor or sensors may be controlled by the user by means of the application installed on his or her mobile telephone.

The generation and transmission of the new firmware by the server and the execution of its installation on the data sensor constitute the updating step S₁₄ of the program, this program being contained in said firmware.

According to the results of the statistical analysis, an update of the program executed on the data sensor is not indispensable to improve the movement recognition.

It then suffices to reconfigure the program according to the configuration parameters obtained further to the statistical analysis step S₁₂. In this case, the configuration parameters are sent by the server 5 to the data sensor 1 via the mobile telephone 3. On reception of these parameters, the data sensor configures the movement recognition program according to the parameters received.

Obtaining and sending the configuration parameters and the application of these parameters to the program of the sensor constitute the configuration step S₁₅.

In a particular embodiment, the server 5 supplies one or more data sensors with a movement classification parameter, said parameter being determined according to analysis parameters, such as the Hamming distance.

In this case, the movement classification parameter constitutes a configuration parameter determined according to the results of the statistical analysis step S₁₂.

Naturally, to meet specific needs, a person competent in the field of the invention will be able to apply modifications to the invention as described above without departing from the scope of the invention. For example, the movement recognition algorithm could be executed by the application installed on the mobile telephone rather than by the program of the data sensor.

The present invention is not limited to the specific embodiments described above, and the modifications which are within the field of application of the present invention will be obvious for a person skilled in the art.

1. A method of data processing configured to improve the movement recognition implemented by a program executed on least one data sensor (1; 1′) within a data processing system, said method comprising the following steps by the data processing system:
collecting raw data (S₄) by said at least one sensor (1);
storing (S₁₀) the raw data in a remote database (7);
performing analysis (S₁₂) of all or part of the raw data stored in the remote database (7);
parameterizing (S₁₄) and/or updating (S₁₅) said program according to the analysis results obtained further to the analyzing step (S₁₂); and
a step of prior testing (S₂), wherein a movement recognition reliability criterion is evaluated if a movement is recognized by the program, such that the other steps of the method are initiated according to the value of the reliability criterion relative to a predetermined threshold.

2. A method according to claim 1, wherein an identifier (I) qualifying the type of movement made by a user of said at least one sensor is associated with said raw data (D) at the collecting step (S₄), the identifier being input manually by the user or automatically based on a predefined sequence of movements to be carried out, the identifier (I) and the data (D) being transmitted to said database (7).

3. A method according to claim 1, wherein said collected raw data (D) are sent by said at least one data sensor (1) to a mobile terminal (3) adapted to transmit said data (D) to the remote database (7).

4. A method according to claim 1, wherein said raw data (D) comprise velocity and/or acceleration inertial data.

5. A method according to claim 1, further comprising a packeting step (S₅), wherein the collected raw data (D) are packeted, so as to provide a structure of data that are calibrated.

6. A method according to claim 1, wherein the parameterizing step (S₁₄) includes the provision to said at least one data sensor (1; 1′) of at least one configuration parameter that is determined according to the results of said analysis (S₁₂).

7. A method according to claim 1, wherein the updating step (S₁₄) includes the provision to said at least one sensor (1; 1′) of a program updated according to the results of said analysis (S₁₂).

8. A method of data processing configured to improve the movement recognition implemented by a program executed on least one data sensor (1; 1′) within a data processing system, said method comprising the following steps by said sensor:
collecting raw data (S₄) by said at least one sensor (1; 1′);
sending the collected raw data to a remote database (7);
receiving at least one configuration parameter and/or an update of said program, said at least one configuration parameter and/or said update being determined according to the results an analysis (S₁₂) of all or part of the raw data stored in the remote database (7); and
a step of prior testing (S₂), wherein a movement recognition reliability criterion is evaluated if a movement is recognized by the program, such that the other steps of the method are initiated according to the value of the reliability criterion relative to a predetermined threshold.

9. A system for data processing configured for improving the movement recognition implemented by a program associated with at least one data sensor (1; 1′), said system comprising:
means for collecting raw data by said at least one sensor;
means for storing the raw data in a remote database (7);
means for analyzing all or part of the raw data stored in the remote database (7); and
means for parameterizing and/or updating said program according to the results obtained by said analysis means.

10. A data sensor configured for improving movement recognition and comprising movement recognition means, said sensor being further comprising:
means for collecting raw data;
means for sending said data to a remote database (7);
means for receiving at least one determined configuration parameter and/or said program configured according to the results an analysis of all or part of the raw data stored in the remote database (7).

11. A data server configured for improving the movement recognition implemented by a program associated with at least one data sensor (1; 1′), said server comprising:
means for receiving raw data obtained by measuring means of at least one data sensor;
means for storing said raw data in a database (7);
means for analyzing all or part of the data contained in said database; and
means for parameterizing and/or updating said program according to the results obtained by said analysis means.

12. (canceled)

13. A non-transitory computer-readable medium on which are stored code instructions of a computer program which, when executed by a computer or microprocessor, performs each of the steps of the method according to claim 1.

14. The method according to claim 2, wherein said collected raw data are sent by said at least one data sensor to a mobile terminal adapted to transmit said data to the remote database.

15. The method according to claim 2, wherein said raw data comprise velocity and/or acceleration inertial data.

16. The method according to claim 3, wherein said raw data comprise velocity and/or acceleration inertial data.

17. The method according to claim 2, further comprising a packeting step, wherein the collected raw data are packeted, so as to provide a structure of data that are calibrated.

18. The method according to claim 3, further comprising a packeting step, wherein the collected raw data are packeted, so as to provide a structure of data that are calibrated.

19. The method according to claim 4, further comprising a packeting step, wherein the collected raw data are packeted, so as to provide a structure of data that are calibrated.

20. The method according to claim 2, wherein the parameterizing step includes the provision to said at least one data sensor of at least one configuration parameter that is determined according to the results of said analysis.

21. The method according to claim 3, wherein the parameterizing step includes the provision to said at least one data sensor of at least one configuration parameter that is determined according to the results of said analysis.
